(19) United States
(12) Patent Application Publication
(10) Pub. No.: US 2026/0183211 A1
(43) Pub. Date: Jul. 2, 2026

(54) ANHYDROUS COSMETIC COMPOSITION COMPRISING BAKUCHIOL

(71) Applicant: AMOREPACIFIC CORPORATION, Seoul (KR)

(72) Inventors: Youngsuk CHO, Yongin-si (KR); Soyoung LEE, Yongin-si (KR); Changgeun YI, Yongin-si (KR)

(73) Assignee: AMOREPACIFIC CORPORATION, Seoul (KR)

(21) Appl. No.: 19/421,266

(22) Filed: Dec. 16, 2025

(30) Foreign Application Priority Data
Dec. 27, 2024 (KR) 10-2024-0198150

(51) Int. Cl. A61K 8/42; A61K 8/34; A61Q 19/08

(52) U.S. Cl. CPC A61K 8/42; A61K 8/347; A61Q 19/08

(57) ABSTRACT

The present invention provides an anhydrous formulation composition comprising bakuchiol and sphingolipid, and a composition for improving skin irritation of bakuchiol comprising sphingolipid as an active ingredient. Specifically, the anhydrous formulation composition of the present invention may improve skin irritation caused by bakuchiol while maintaining excellent effects of bakuchiol by including sphingolipid.

### CROSS REFERENCE TO RELATED APPLICATION

The present application claims priority to Korean Patent Application No. 10-2024-0198150, filed Dec. 27, 2024, the entire contents of which are hereby incorporated by this reference.

### BACKGROUND OF THE INVENTION

### Field of the Invention

One aspect of the present disclosure is an anhydrous cosmetic composition comprising bakuchiol.

### Description of the Related Art

Bakuchiol is known as a natural plant-derived ingredient that may improve skin while providing antioxidant and anti-aging effects similar to retinol, and at the same time without irritation or dryness that retinol has. In addition, bakuchiol helps reduce wrinkles and fine lines by promoting collagen production and accelerating skin cell turnover.

However, invention content using sphingolipid in an anhydrous formulation for improving stability of bakuchiol is absent.

The present inventors sought to secure stability of an anhydrous cosmetic composition comprising oil and sphingolipid, and at the same time to meet needs of sensitive-skin consumers through alleviation of irritation level of bakuchiol.

### SUMMARY OF THE INVENTION

In one aspect, the present invention is directed to providing an anhydrous formulation composition comprising bakuchiol and sphingolipid.

In another aspect, the present invention is directed to providing a composition for improving skin irritation of bakuchiol comprising sphingolipid as an active ingredient.

In one aspect, the present invention provides an anhydrous formulation composition comprising bakuchiol and sphingolipid.

In one exemplary embodiment, a melting point of the sphingolipid may be 40° C. to 70° C.

In one exemplary embodiment, the bakuchiol may be included in an amount of 0.01 wt % to 5 wt % based on a total weight of the composition.

In one exemplary embodiment, the bakuchiol and the sphingolipid may be included in an amount of 0.4 wt % to 10 wt % based on the total weight of the composition.

In one exemplary embodiment, the bakuchiol and the sphingolipid may be included in the composition in a weight ratio of 1:0.05 to 10.

In one exemplary embodiment, the sphingolipid may be one or more selected from a group consisting of ceramide, a derivative thereof, and an analogue thereof.

In one exemplary embodiment, the ceramide may be Ceramide NP or Hydroxypropyl Bislauramide MEA.

In one exemplary embodiment, the composition may further include an oil.

In one exemplary embodiment, the oil may be one or more selected from a group consisting of hydrocarbon oil, ester oil, ether oil, and natural oil.

In one exemplary embodiment, the oil may be squalane or cetyl ethylhexanoate.

In one exemplary embodiment, the composition may be for skin improvement.

In one exemplary embodiment, the skin improvement may be one or more selected from a group consisting of skin antioxidation, skin anti-aging, promotion of collagen production in skin, promotion of skin turnover, and improvement of skin wrinkles.

In one exemplary embodiment, the composition may be for cosmetic use.

In another aspect, the present invention provides a composition for improving skin irritation of bakuchiol comprising sphingolipid as an active ingredient.

In one exemplary embodiment, a melting point of the sphingolipid may be 40° C. to 70° C.

In one exemplary embodiment, the bakuchiol and the sphingolipid may be included in the composition in a weight ratio of 1:0.05 to 10.

In one exemplary embodiment, the sphingolipid may be one or more selected from a group consisting of ceramide, a derivative thereof, and an analogue thereof.

In one exemplary embodiment, the ceramide may be Ceramide NP or Hydroxypropyl Bislauramide MEA.

In one exemplary embodiment, the composition may further include an oil.

In one exemplary embodiment, the oil may be one or more selected from a group consisting of hydrocarbon oil, ester oil, ether oil, and natural oil.

In one aspect, a composition of the present invention has excellent skin improvement effects.

In one aspect, a composition of the present invention may help skin antioxidation, skin anti-aging, promotion of collagen production in skin, promotion of skin turnover, and improvement of skin wrinkles by including bakuchiol.

In one aspect, a composition of the present invention may alleviate skin irritation of bakuchiol by including sphingolipid.

In one aspect, a composition of the present invention has excellent formulation stability.

In one aspect, a composition of the present invention provides low irritation to skin.

### BRIEF DESCRIPTION OF THE DRAWINGS

FIG. 1 is a photograph comparing formulation stability of one embodiment of the present invention and comparative examples.

FIGS. 2A and 2B show microscope images of Comparative Example 3 (FIG. 2A: polarized light, FIG. 2B: daylight).

FIGS. 3A and 3B show microscope images of Example 1 (FIG. 3A: polarized light, FIG. 3B: daylight).

FIG. 4 is a DSC data analysis result of Hydroxypropyl Bispalmitamide MEA used in Comparative Example 4.

FIG. 5 is a DSC data analysis result of Hydroxypropyl Bislauramide MEA used in Example 3.

FIG. 6 is a DSC data analysis result of Sphingolipid (O-cera) used in Example 2.

### DETAILED DESCRIPTION OF THE INVENTION

The terms used in the present specification are selected from general terms currently widely used in the art in consideration of functions in the present disclosure, but the terms may vary according to the intention of those skilled in the art, precedents, the emergence of new technology, or the like in the art. Further, specified terms are selected arbitrarily by the applicant, and in this case, the detailed meaning thereof will be described in the detailed description of the invention. Thus, the terms used in the present specification should be defined based not on simple names but on the meaning of the terms and the overall description of the present disclosure.

Unless otherwise defined, all terms used herein, including technical or scientific terms, have the same meaning as commonly understood by those skilled in the art to which the present disclosure pertains. The terms which are commonly understood should be interpreted as having meanings consistent with meanings in the context of related technologies and should not be interpreted as ideal or excessively formal meanings unless explicitly defined in the present disclosure.

Numerical value ranges are inclusive of the values defined in the present disclosure. Every maximum numerical limitation given throughout the present specification includes every lower numerical limitation, as if such lower numerical limitations were expressly written. Every minimum numerical limitation given throughout the present specification includes every higher numerical limitation, as if such higher numerical limitations were expressly written. Every numerical limitation given throughout the present specification will include every better numerical range within the broader numerical range, as if the narrower numerical limitations were expressly written.

As used in the present specification, the words “comprising”, “having”, “including” are inclusive or open-ended and do not exclude additional unrecited elements or method steps. The term “or combinations thereof” used in the present specification refers to all permutations and combinations of the items listed preceding the term. For example, “A, B, C, or combinations thereof” is intended to be A, B, C, AB, AC, BC, or ABC, and to include at least one of BA, CA, CB, CBA, BCA, ACB, BAC or CAB, where order is important in a particular context. With the example above, “A, B, C, or combinations thereof” may include combinations containing repetitions of one or more items or terms, such as BB, AAA, MB, BBC, AAABCCCC, CBBAAA, CABABB, and the like. Those skilled in the art will understand that there is typically no limit to the number of items or terms in any combination, unless the context clearly indicates otherwise.

Hereinafter, exemplary embodiments of the present disclosure are described in detail.

In one aspect, the present invention provides an anhydrous formulation composition comprising bakuchiol and sphingolipid.

The bakuchiol is an organic compound having a structure of meroterpene of a terpenol-phenol series and is an ingredient having various efficacies such as anti-inflammatory, antioxidant, antitumor, and antibacterial activities. Bakuchiol may perform a role similar to retinol, and retinol is an ingredient known to be effective for fine wrinkles, wrinkles, skin texture, skin tone, moisture, pigmentation, acne, and the like as an anti-aging raw material, but has disadvantages as a cosmetic due to skin irritation such as stinging and dermatitis.

The sphingolipid means a complex of lipids in which sphingosine or its related base, a polar head portion, and a long-chain saturated fatty acid or a long-chain fatty acid having a single degree of unsaturation linked to an amino group of sphingosine are connected. In addition, the sphingolipid refers to complex long-chain lipids (fats) that act as skin-replenishing and conditioning ingredients and may help maintain an intact skin barrier and prevent moisture loss to cultivate healthier, smoother, and younger-looking skin. Sphingolipid is a precursor of ceramide and may help produce more ceramide which is important for maintaining strong skin.

The anhydrous formulation means a formulation that does not contain water.

In one exemplary embodiment, a melting point of the sphingolipid may be 40° C. to 70° C. The inventors of the present invention have confirmed that, by including the sphingolipid having a melting point within a specific range of one embodiment in the composition, even when bakuchiol is included at a high content of 0.3% or more in the composition, a skin irritation level due to bakuchiol may be remarkably improved. For example, the melting point of the sphingolipid may be 40° C. or more, 42° C. or more, 44° C. or more, 46° C. or more, 48° C. or more, 50° C. or more, 52° C. or more, 54° C. or more, 56° C. or more, 58° C. or more, 60° C. or more, 62° C. or more, 64° C. or more, 66° C. or more, 68° C. or more, 70° C. or more, 72° C. or more, 74° C. or more, 76° C. or more, or 78° C. or more, but is not limited thereto. In addition, the melting point of the sphingolipid may be 80° C. or less, 78° C. or less, 76° C. or less, 74° C. or less, 72° C. or less, 70° C. or less, 68° C. or less, 66° C. or less, 64° C. or less, 62° C. or less, 60° C. or less, 58° C. or less, 56° C. or less, 54° C. or less, 52° C. or less, 50° C. or less, 48° C. or less, 46° C. or less, 44° C. or less, or 42° C. or less, but is not limited thereto.

In one exemplary embodiment, the bakuchiol may be included in an amount of 0.01 wt % to 5 wt % based on a total weight of the composition. For example, the bakuchiol may be included in an amount of 0.01 wt % or more, 0.05 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, or 0.9 wt % or more based on the total weight of the composition, but is not limited thereto. In addition, the bakuchiol may be included in an amount of 5 wt % or less, 4.8 wt % or less, 4.6 wt % or less, 4.4 wt % or less, 4.2 wt % or less, 4 wt % or less, 3.8 wt % or less, 3.6 wt % or less, 3.4 wt % or less, 3.2 wt % or less, 3 wt % or less, 2.8 wt % or less, 2.6 wt % or less, 2.4 wt % or less, 2.2 wt % or less, 2 wt % or less, 1.8 wt % or less, 1.6 wt % or less, 1.5 wt % or less, 1.4 wt % or less, 1.3 wt % or less, 1.2 wt % or less, or 1.1 wt % or less based on the total weight of the composition, but is not limited thereto.

In one exemplary embodiment, the bakuchiol and the sphingolipid may be included in an amount of 0.4 wt % to 10 wt % based on the total weight of the composition. When bakuchiol is included in a high content in the composition, it may cause skin irritation when applied to the skin. The inventors of the present invention have confirmed that the skin irritation caused by bakuchiol may be alleviated by including sphingolipid in an anhydrous formulation. For example, the bakuchiol and the sphingolipid may be included in an amount of 0.4 wt % or more, 0.6 wt % or more, 0.8 wt % or more, 1 wt % or more, 1.1 wt % or more, 1.2 wt % or more, 1.3 wt % or more, 1.4 wt % or more, 1.5 wt % or more, 1.6 wt % or more, 1.7 wt % or more, 1.8 wt % or more, or 1.9 wt % or more based on the total weight of the composition, but is not limited thereto. In addition, the bakuchiol and the sphingolipid may be included in an amount of 10 wt % or less, 9.5 wt % or less, 9 wt % or less, 8.5 wt % or less, 8 wt % or less, 7.5 wt % or less, 7 wt % or less, 6.5 wt % or less, 6 wt % or less, 5.5 wt % or less, 5 wt % or less, 4.8 wt % or less, 4.6 wt % or less, 4.4 wt % or less, 4.2 wt % or less, 4 wt % or less, 3.8 wt % or less, 3.6 wt % or less, 3.4 wt % or less, 3.2 wt % or less, 3 wt % or less, 2.9 wt % or less, 2.8 wt % or less, 2.7 wt % or less, 2.6 wt % or less, 2.5 wt % or less, 2.4 wt % or less, 2.3 wt % or less, 2.2 wt % or less, or 2.1 wt % or less based on the total weight of the composition, but is not limited thereto.

In one exemplary embodiment, the bakuchiol and the sphingolipid may be included in the composition in a weight ratio of 1:0.05 to 10. For example, the weight ratio of the bakuchiol and the sphingolipid may be included in the composition at a weight ratio of 0.05 or more, 0.1 or more, 0.2 or more, 0.3 or more, 0.4 or more, 0.5 or more, 0.6 or more, 0.7 or more, 0.75 or more, 0.8 or more, 0.85 or more, 0.9 or more, 0.95 or more, 1 or more, 1.05 or more, 1.1 or more, 1.15 or more, 1.2 or more, 1.25 or more, 1.3 or more, 1.35 or more, 1.4 or more, 1.45 or more, 1.5 or more, 1.6 or more, 1.7 or more, 1.8 or more, 1.9 or more, 2 or more, 2.5 or more, 3 or more, 3.5 or more, 4 or more, or 4.5 or more based on 1 of bakuchiol, but is not limited thereto. In addition, the weight ratio of the bakuchiol and the sphingolipid may be included in the composition at a weight ratio of 10 or less, 9 or less, 8 or less, 7 or less, 6 or less, 5 or less, 4 or less, 3 or less, 2 or less, 1.9 or less, 1.8 or less, 1.7 or less, 1.6 or less, 1.5 or less, 1.45 or less, 1.4 or less, 1.35 or less, 1.3 or less, 1.25 or less, 1.2 or less, 1.15 or less, 1.1 or less, 1.05 or less, 1 or less, 0.95 or less, 0.9 or less, 0.85 or less, 0.8 or less, 0.75 or less, 0.7 or less, 0.65 or less, 0.6 or less, 0.5 or less, 0.4 or less, 0.3 or less, 0.2 or less, or 0.1 or less based on 1 of bakuchiol, but is not limited thereto.

In one exemplary embodiment, the sphingolipid may be one or more selected from a group consisting of ceramide, a derivative thereof, and an analogue thereof.

The ceramide means a molecule that, as one type of sphingolipid, is composed of sphingosine and a fatty acid.

In one exemplary embodiment, the ceramide may be Ceramide NP or Hydroxypropyl Bislauramide MEA.

In one exemplary embodiment, the composition may further include an oil.

In one exemplary embodiment, the oil may be one or more selected from a group consisting of hydrocarbon oil, ester oil, ether oil, and natural oil. For example, the oil may not be a silicone-based oil, but is not limited thereto.

In one exemplary embodiment, the oil may be squalane or cetyl ethylhexanoate.

In one exemplary embodiment, the composition may be for skin improvement.

In one exemplary embodiment, the skin improvement may be one or more selected from a group consisting of skin antioxidation, skin anti-aging, promotion of collagen production in skin, promotion of skin turnover, and improvement of skin wrinkles.

In one exemplary embodiment, the composition may be for cosmetic use.

An appearance of the cosmetic composition may contain a medium or base that is cosmetically or dermatologically acceptable. This may be any formulation suitable for topical application, for example, a solution, a gel, a solid, a paste-type anhydrous product, an emulsion obtained by dispersing an oil phase in an aqueous phase, a suspension, a microemulsion, a microcapsule, fine granules, or a dispersion of vesicles of ionic (liposome) or nonionic type, or may be provided in the form of a cream, toner, lotion, powder, ointment, spray, or concealer stick. These compositions may be manufactured according to conventional methods in the relevant field.

The cosmetic composition is not particularly limited in its formulation and may be formulated as cosmetics such as softening toner, astringent toner, nutritive toner, nutritive cream, massage cream, essence, eye cream, eye essence, cleansing cream, cleansing foam, cleansing water, facial pack, powder, body lotion, body cream, body oil, or body essence.

When the formulation of the cosmetic composition is paste, cream, or gel, carrier ingredients such as animal fibers, plant fibers, wax, paraffin, starch, tragacanth, cellulose derivatives, polyethylene glycol, silicone, bentonite, silica, talc, or zinc oxide may be used.

When the formulation of the cosmetic composition is powder or spray, carrier ingredients such as lactose, talc, silica, aluminum hydroxide, calcium silicate, or polyamide powder may be used, and particularly in the case of a spray, may additionally include a propellant such as chlorofluorohydrocarbon, propane/butane, or dimethyl ether.

The cosmetic composition may further include functional additives and ingredients included in general cosmetic compositions. The functional additive may include one or more ingredients selected from a group consisting of water-soluble vitamins, oil-soluble vitamins, polymer peptides, polymer polysaccharides, sphingolipids, and seaweed extracts.

In the cosmetic composition, in addition to the functional additive, ingredients included in general cosmetic compositions may be blended as necessary. Other blending ingredients to be included may include oily ingredients, moisturizers, emollients, organic and inorganic pigments, organic powders, preservatives, bactericides, antioxidants, plant extracts, pH adjusters, alcohols, colorants, fragrances, blood circulation promoters, cooling agents, antiperspirants, purified water, and the like.

In another aspect, the present invention provides a composition for improving skin irritation of bakuchiol comprising sphingolipid as an active ingredient.

The sphingolipid and the bakuchiol are as described above.

In one exemplary embodiment, a melting point of the sphingolipid may be 40° C. to 70° C. For example, the melting point of the sphingolipid may be 40° C. or more, 42° C. or more, 44° C. or more, 46° C. or more, 48° C. or more, 50° C. or more, 52° C. or more, 54° C. or more, 56° C. or more, 58° C. or more, 60° C. or more, 62° C. or more, 64° C. or more, 66° C. or more, 68° C. or more, 70° C. or more, 72° C. or more, 74° C. or more, 76° C. or more, or 78° C. or more, but is not limited thereto. In addition, the melting point of the sphingolipid may be 80° C. or less, 78° C. or less, 76° C. or less, 74° C. or less, 72° C. or less, 70° C. or less, 68° C. or less, 66° C. or less, 64° C. or less, 62° C. or less, 60° C. or less, 58° C. or less, 56° C. or less, 54° C. or less, 52° C. or less, 50° C. or less, 48° C. or less, 46° C. or less, 44° C. or less, or 42° C. or less, but is not limited thereto.

In one exemplary embodiment, the bakuchiol and the sphingolipid may be included in the composition in a weight ratio of 1:0.05 to 10. For example, the weight ratio of the bakuchiol and the sphingolipid may be included in the composition at a weight ratio of 0.05 or more, 0.1 or more, 0.2 or more, 0.3 or more, 0.4 or more, 0.5 or more, 0.6 or more, 0.7 or more, 0.75 or more, 0.8 or more, 0.85 or more, 0.9 or more, 0.95 or more, 1 or more, 1.05 or more, 1.1 or more, 1.15 or more, 1.2 or more, 1.25 or more, 1.3 or more, 1.35 or more, 1.4 or more, 1.45 or more, 1.5 or more, 1.6 or more, 1.7 or more, 1.8 or more, 1.9 or more, 2 or more, 2.5 or more, 3 or more, 3.5 or more, 4 or more, or 4.5 or more based on 1 of bakuchiol, but is not limited thereto. In addition, the weight ratio of the bakuchiol and the sphingolipid may be included in the composition at a weight ratio of 10 or less, 9 or less, 8 or less, 7 or less, 6 or less, 5 or less, 4 or less, 3 or less, 2 or less, 1.9 or less, 1.8 or less, 1.7 or less, 1.6 or less, 1.5 or less, 1.45 or less, 1.4 or less, 1.35 or less, 1.3 or less, 1.25 or less, 1.2 or less, 1.15 or less, 1.1 or less, 1.05 or less, 1 or less, 0.95 or less, 0.9 or less, 0.85 or less, 0.8 or less, 0.75 or less, 0.7 or less, 0.65 or less, 0.6 or less, 0.5 or less, 0.4 or less, 0.3 or less, 0.2 or less, or 0.1 or less based on 1 of bakuchiol, but is not limited thereto.

In one exemplary embodiment, the sphingolipid may be one or more selected from a group consisting of ceramide, a derivative thereof, and an analogue thereof. The ceramide is as described above.

In one exemplary embodiment, the ceramide may be Ceramide NP or Hydroxypropyl Bislauramide MEA.

In one exemplary embodiment, the composition may further include an oil.

In one exemplary embodiment, the oil may be one or more selected from a group consisting of hydrocarbon oil, ester oil, ether oil, and natural oil.

In one exemplary embodiment, the oil may be squalane or cetyl ethylhexanoate.

### Manufacturing Example

An anhydrous solution including bakuchiol and sphingolipid was manufactured according to the composition (wt %) shown in Table 1 below. After heating the oil used as a solvent to 70° C., the sphingolipid, ceramide derivative, or analogue was added and completely dissolved, and then bakuchiol was added. During heating, dissolving, and dispersing processes, mixing and blending were performed at 300 RPM using an Agi mixer (Shinwon ENG, SWAT-01).

TABLE 1

| | | | | | Compar- | Compar- | Compar- | Compar- | Compar- | Compar- |
|---|---|---|---|---|---|---|---|---|---|---|
| Name of Raw | | | | | ative | ative | ative | ative | ative | ative |
| material | Example 1 | Example 2 | Example 3 | Example 4 | Example 1 | Example 2 | Example 3 | Example 4 | Example 5 | Example 6 |
| Bakuchiol | 1 | 1 | 1 | 3 | 0.5 | 1 | 1 | 1 | 1 | 1 |
| Squalane | To 100 | | To 100 | To 100 | To 100 | To 100 | To 100 | To 100 | To 100 | To 100 |
| Cetyl | | To 100 | | | | | | | | |
| Ethylhexanoate | | | | | | | | | | |
| O-cera * | 1 | 1 | | 1 | | | | | | |
| (Sphingolipids | | | | | | | | | | |
| (Acetylation of | | | | | | | | | | |
| ceramide NP), | | | | | | | | | | |
| Ceramide NP) | | | | | | | | | | |
| Hydroxypropyl | | | 1 | | | | | | | |
| Bislauramide MEA | | | | | | | | | | |
| (PC-102) | | | | | | | | | | |
| CETYL-PG | | | | | | | 1 | | | |
| HYDROXYETHYL | | | | | | | | | | |
| PALMITAMIDE | | | | | | | | | | |
| (Ceramoist **) | | | | | | | | | | |
| Hydroxypropyl | | | | | | | | 1 | | |
| Bispalmitamide MEA | | | | | | | | | | |
| (PC-104) | | | | | | | | | | |
| Ceramide NP *** | | | | | | | | | 1 | |
| Ceramide EOP **** | | | | | | | | | | 1 |

* O-cera uses DS-O CERA from Croda Korea (DS-O CERA consists of 95-99 wt % sphingolipids and 1-5 wt % Ceramide NP),
** Ceramoist is supplied by Dongguan Huagong Chuawei Biotechnologies Co., Ltd.
*** Ceramide NP uses DS-CERAMIDE Y3O from Croda Korea,
**** Ceramide EOP uses DS-Ceramide EOP from Croda Korea

The melting points of the sphingolipids used in Examples 1 to 3 and Comparative Examples 1 to 6 are shown in Table 2 below.

TABLE 2

| | Melting |
|---|---|
| | Point |
| Type of Sphingolipid | (° C.) |
| O-cera | 40-50 |
| (Sphingolipids (Acetylation of ceramide NP), Ceramide NP) | |
| Hydroxypropyl Bislauramide MEA | 55.29 |
| CETYL-PG HYDROXYETHYL PALMITAMIDE | 76.74 |
| Hydroxypropyl Bispalmitamide MEA | 70.45 |
| Ceramide NP | 98-110 |
| Ceramide EOP | 88-96 |

### Experimental Example

### 1. Formulation Stability Comparison Experiment

Examples 1 to 3 and Comparative Examples 1 to 6 were compared in formulation stability in a manner of storing them for 4 weeks under freezing, refrigeration, room temperature, 45° C., and CYC constant-temperature bath conditions, and the experimental results are as shown in Table 3 below.

TABLE 3

| | | | | | Comparative | Comparative | Comparative | Comparative | Comparative | Comparative |
|---|---|---|---|---|---|---|---|---|---|---|
| Classification | Example 1 | Example 2 | Example 3 | Example 4 | Example 1 | Example 2 | Example 3 | Example 4 | Example 5 | Example 6 |
| Formulation | Good | Good | Good | Good | Good | Good | Poor | Poor | Poor | Poor |
| Stability | | | | | | | | | | |

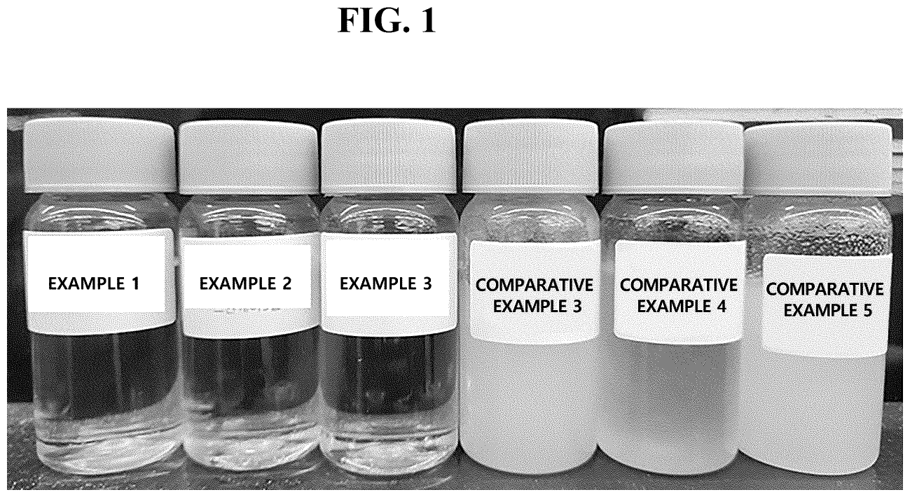

Specifically, Examples 1 to 3 and Comparative Examples 1 and 2 were confirmed to be maintained consistently with the initial appearance quality at the time of manufacture consistently in all cases of storage under freezing, refrigeration, room temperature, 45° C., and CYC constant-temperature bath conditions for 4 weeks (FIG. 1).

In contrast, in the case of Comparative Examples 3 to 6, it was confirmed that, when stored at room temperature after manufacture, crystallization occurred from the day after manufacture (FIG. 1), and that crystallization occurred within 1 week in all cases of storage under freezing, refrigeration, CYC constant-temperature bath, and room temperature conditions, and that crystallization progressed from 1 week of storage under the high-temperature condition of 45° C.

### 2. Characteristic Analysis of Examples and Comparative Examples

### 1) Characteristic Analysis Using Microscope Images

Comparative Example 3 and Example 1 were photographed at 100× magnification using a microscope (Ni-U Somple pol+DS-Ri2, Nikon).

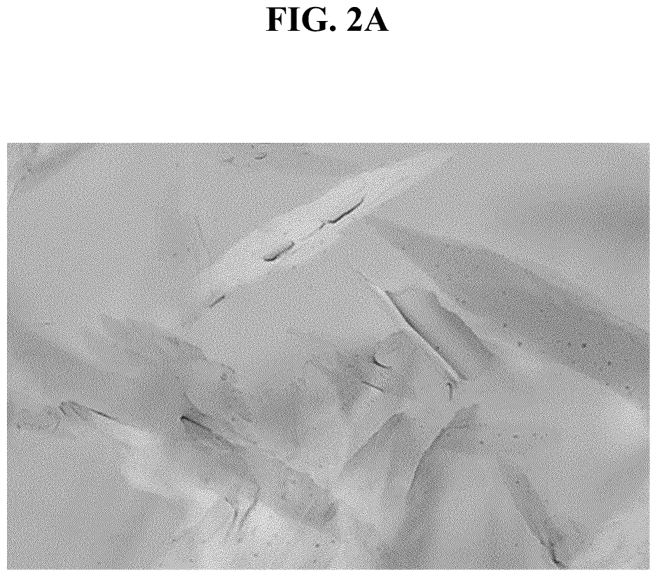

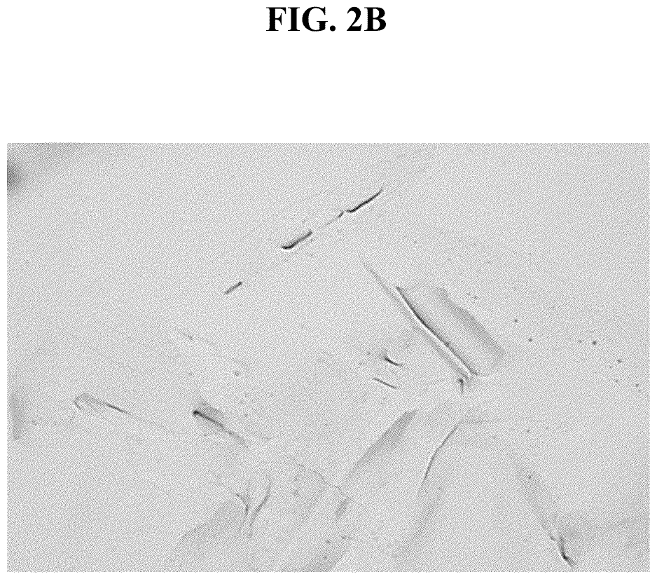

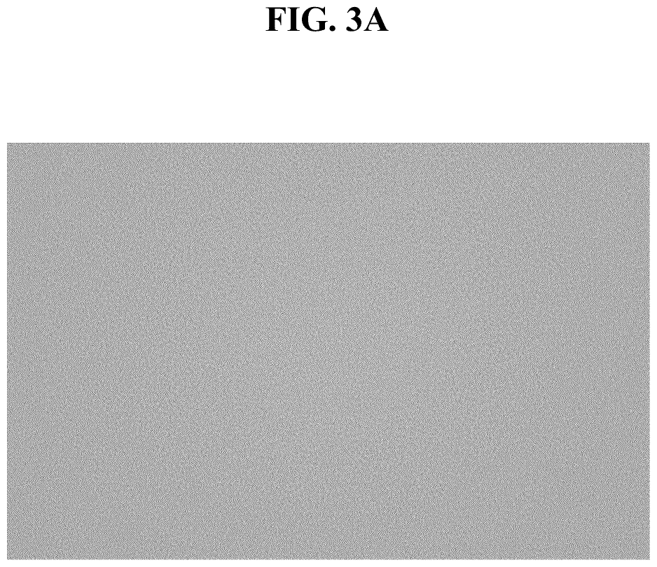

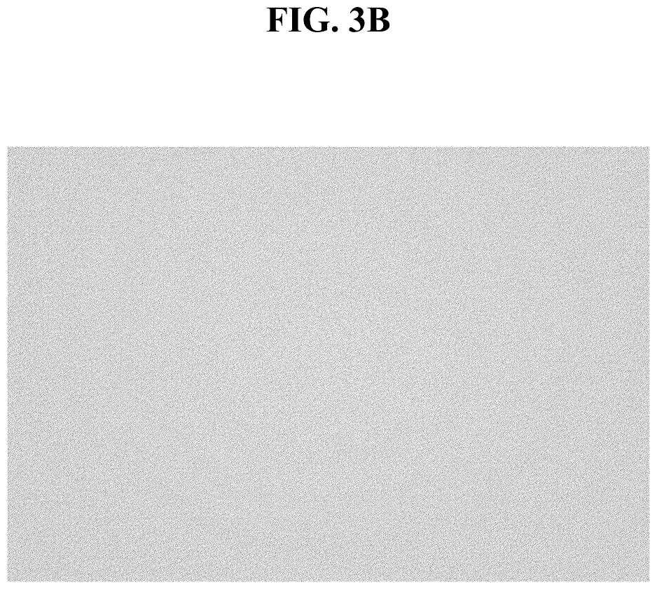

As a result of the experiment, Comparative Example 3 showed that crystallization proceeded and ceramide was precipitated, and non-uniform images in which light was refracted or were non-uniform were observed (FIG. 2A: polarized light, FIG. 2B: daylight). In contrast, Example 1 maintained a smooth formulation without crystallization, and it was confirmed that light was not refracted and no color change was observed in the image (FIG. 3A: polarized light, FIG. 3B: daylight).

### 2) Characteristic Analysis Using Differential Scanning Calorimetry

DSC data analysis was performed for Hydroxypropyl Bispalmitamide MEA used in Comparative Example 4, Hydroxypropyl Bislauramide MEA used in Example 3, and Sphingolipid (O-cera) used in Example 2 using a Thermal Analysis System (Discovery DSC 250, TA Instruments).

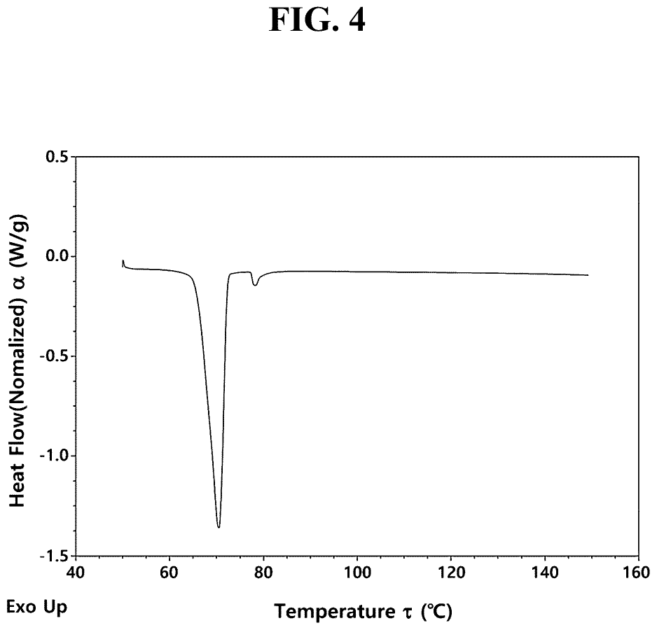

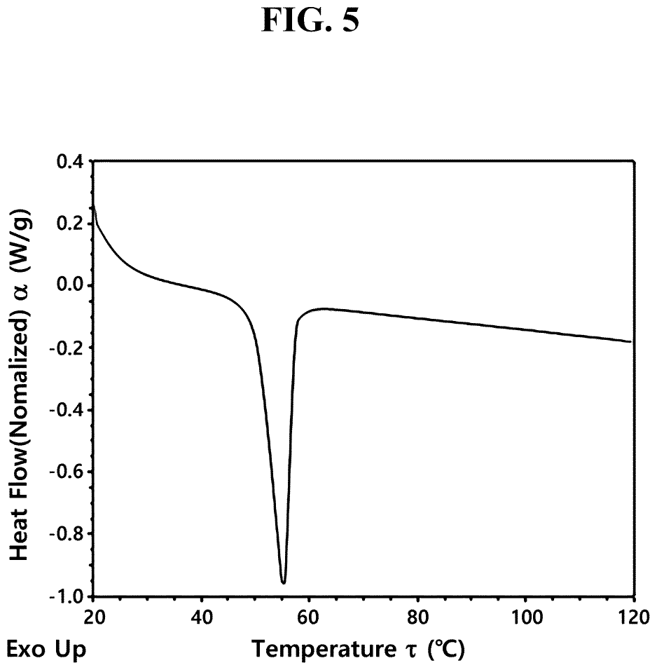

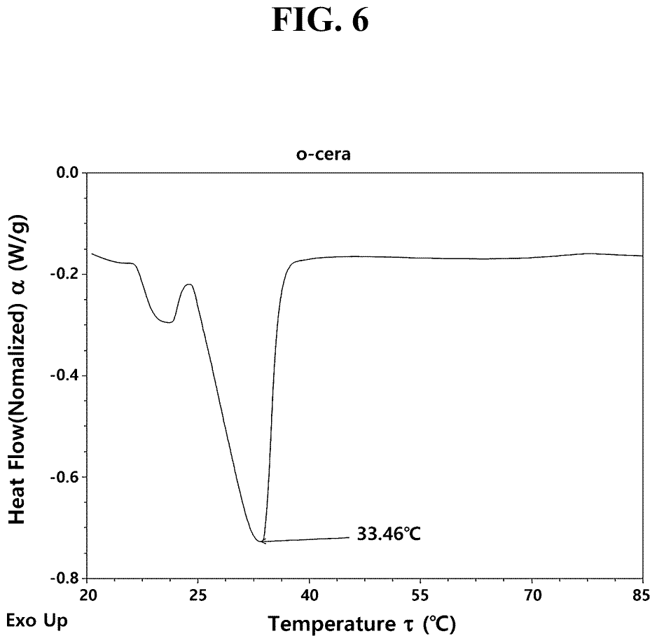

As a result of the experiment, it was confirmed that the peak of Hydroxypropyl Bispalmitamide MEA occurred near 70° C. (FIG. 4), the peak of Hydroxypropyl Bislauramide MEA occurred near 55° C. (FIG. 5), and the peak of Sphingolipid (O-cera) occurred near 33° C. (FIG. 6).

Through the above experimental results, crystallization temperature ranges of each ingredient were identified. In the case of Hydroxypropyl Bispalmitamide MEA having a crystallization peak at 70° C., it was confirmed that crystallization was easily induced when stored at room temperature in hydrocarbon-based oil and that it was inapplicable to development of a cosmetic of such a type. In the case of Hydroxypropyl Bislauramide MEA and O-cera, which are ingredients having crystallization peaks below 70° C., it may exhibit an effect of having a relatively favorable appearance state.

### 3. Sensory Evaluation Related to Irritation-Alleviation Efficacy

To evaluate the irritation-alleviation efficacy of bakuchiol of the composition of the present invention, forty evaluators in their 20 s to 40 s were each instructed to evaluate the item of irritation-alleviation degree.

The experimental method is as follows.

After attaching a patch of the sample to the upper back of the subjects and removing the patch after 24 hours, a first reading is performed 30 minutes later, and a second reading is performed after 24 hours. The skin irritation of the sample was determined by obtaining an average skin response value by assigning weighting according to the degree of positive reaction on the skin. The test was evaluated in stages, and the test was performed based on the terminology presented in Table 4 below, which is a modified test criterion from the ICDRG guideline, the CTFA guideline (1981), and the test criteria of Frosch & Kligman (1979), and the test results were calculated as the average value of responses for each subject.

TABLE 4

| Classification | No visible reaction |
|---|---|
| 1+ | Slight erythema, either spotty or diffuse |
| 2+ | Moderate uniform erythema |
| 3+ | Intense erythema with edema |
| 4+ | Intense erythema with edema & vesicles |

For comparison, a sensory evaluation was conducted for primary irritation according to the concentration of bakuchiol in cases where sphingolipid was not included, and as a result of the experiment, it was confirmed that skin irritation pattern caused by bakuchiol increased in a concentration-dependent manner (Table 5).

TABLE 5

| Name of Raw | Comparative | Comparative | Comparative |
|---|---|---|---|
| material | Example 1 | Example 7 | Example 8 |
| Bakuchiol | 0.5 | 1.5 | 2.5 |
| Squalane | To 100 | To 100 | To 100 |
| Irritation | 1.22 | 1.83 | 2.44 |
| Evaluation | | | |

Next, a sensory evaluation was conducted by using Examples 1 to 3, which include the sphingolipid, and Comparative Examples 1 and 2 as samples (Comparative Examples 3 to 6 were not subjected to sensory evaluation because the formulations were not stable). Meanwhile, when the irritation level was 1 or more, it was described as slight irritation.

TABLE 6

| | | | | | Comparative | Comparative | Comparative | Comparative | Comparative | Comparative |
|---|---|---|---|---|---|---|---|---|---|---|
| Classification | Example 1 | Example 2 | Example 3 | Example 4 | Example 1 | Example 2 | Example 3 | Example 4 | Example 5 | Example 6 |
| Primary | No | No | No | No | Slight | Slight | — | — | — | — |
| Irritation | irritation | irritation | irritation | irritation | irritation | irritation | | | | |

As can be confirmed from Table 6, Examples 1 to 3, in which compositions including the sphingolipid having a melting point of 40° C. to 70° C. were applied, were evaluated as no irritation, whereas compositions including the sphingolipid having melting points outside the above range were evaluated as showing slight irritation.

What is claimed is:

1. An anhydrous formulation composition comprising bakuchiol and sphingolipid.

2. The anhydrous formulation composition of claim 1, wherein a melting point of the sphingolipid is 40° C. to 70° C.

3. The anhydrous formulation composition of claim 1, wherein the bakuchiol is included in an amount of 0.01 wt % to 5 wt % based on a total weight of the composition.

4. The anhydrous formulation composition of claim 1, wherein the bakuchiol and the sphingolipid are included in an amount of 0.4 wt % to 10 wt % based on a total weight of the composition.

5. The anhydrous formulation composition of claim 1, wherein the bakuchiol and the sphingolipid are included in the composition in a weight ratio of 1:0.05 to 10.

6. The anhydrous formulation composition of claim 1, wherein the sphingolipid is one or more selected from a group consisting of ceramide, a derivative thereof, and an analogue thereof.

7. The anhydrous formulation composition of claim 6, wherein the ceramide is Ceramide NP or Hydroxypropyl Bislauramide MEA.

8. The anhydrous formulation composition of claim 1, wherein the composition further includes an oil.

9. The anhydrous formulation composition of claim 8, wherein the oil is one or more selected from a group consisting of hydrocarbon oil, ester oil, ether oil, and natural oil.

10. The anhydrous formulation composition of claim 8, wherein the oil is squalane or cetyl ethylhexanoate.

11. The anhydrous formulation composition of claim 1, wherein the composition is for cosmetic use.

12. A method for improving skin by applying to skin an effective amount of the anhydrous formulation composition of claim 1.

13. The method of claim 12, wherein the skin improvement is one or more selected from a group consisting of skin antioxidation, skin anti-aging, promotion of collagen production in skin, promotion of skin turnover, and improvement of skin wrinkles.

14. A method for improving skin irritation caused by bakuchiol by applying to skin a composition comprising an effective amount of sphingolipid.

15. The method of claim 14, wherein a melting point of the sphingolipid is 40° C. to 70° C.

16. The method of claim 14, wherein the bakuchiol and the sphingolipid are included in the composition in a weight ratio of 1:0.05 to 10.

17. The method of claim 14, wherein the sphingolipid is one or more selected from a group consisting of ceramide, a derivative thereof, and an analogue thereof.

18. The method of claim 17, wherein the ceramide is Ceramide NP or Hydroxypropyl Bislauramide MEA.

19. The method of claim 14, wherein the composition further includes an oil.

20. The method of claim 19, wherein the oil is one or more selected from a group consisting of hydrocarbon oil, ester oil, ether oil, and natural oil.
